(19) United States
(12) Patent Application Publication
(10) Pub. No.: US 2008/0274074 A1
(43) Pub. Date: Nov. 6, 2008

(54) Method for Controlled Free Radical Polymerization or Copolymerization of Methacrylic and/or Methacrylate Monomers or Exclusively Methacrylic and/or Methacrylate Copolymers

(75) Inventors: Jean-Luc Couturier, Lyon (FR); Yohann Guillaneuf, Marseille (FR); Denis Bertin, Marseille (FR); Didior Gigmes, Marseille (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Colombes (FR)

(21) Appl. No.: 12/094,696

(22) PCT Filed: Nov. 24, 2006
(86) PCT No.: PCT/FR2006/051231

§ 371 Date: May 22, 2008

(30) Foreign Application Priority Data
Nov. 24, 2005 (FR) 0553590

(51) Int. Cl. A61K 8/84; C08G 63/44; C08G 63/66; C08G 73/10; C08G 69/02

(52) U.S. Cl. 424/78.03; 528/363; 528/361; 528/27; 528/322; 528/327; 528/332

(57) ABSTRACT

The invention relates to a method for the polymerization of one or more methacrylate and/or methacrylic monomers, for the synthesis of methacrylate or methacrylic polymers or of exclusively methacrylic and/or methacrylate copolymers, comprising a step of bringing said monomer(s) into contact with one at least of the compounds of formula (I) and (II) below:

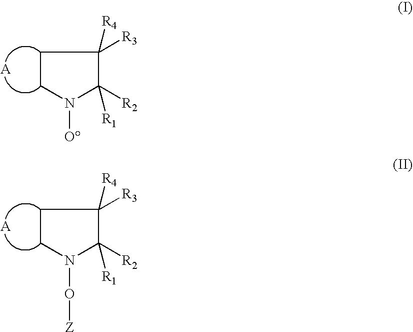

in which:
R₁ and R₂ may represent a phenyl group, R₃ and R₄ may together form an ═N-phenyl group, Z represents a group of formula —CR₈R₉R₁₀, in which R₈ and R₉ may represent methyl groups and R₁₀ may represent a —COO-phenyl group.

### TECHNICAL FIELD

The present invention relates to a method for the controlled radical polymerization or copolymerization of methacrylic and/or methacrylate monomers and in particular of methyl methacrylate, to produce methacrylate or methacrylic polymers or exclusively methacrylic and/or methacrylate copolymers.

The general field of the invention is therefore that of controlled radical polymerization.

Controlled radical polymerization makes it possible to reduce the deactivation reactions of the growing radical species, in particular the termination step, reactions which, in conventional radical polymerization, interrupt the growth of the polymer chain irreversibly and without control.

In order to decrease the probability of the termination reactions, it has been proposed to transiently and reversibly block the growing radical species, by forming active species known as “dormant species” in the form of a bond having a low dissociation energy. This thus makes it possible to restart the polymerization and thus to obtain better Degree of conversions, a controlled number (or weight) average molecular weight and a lower polydispersity index than in conventional radical polymerization. This also makes it possible to synthesize block copolymers by starting the synthesis of a block on the dormant species.

Recently, trials to synthesize methacrylate polymers by the controlled radical route have been carried out.

Thus, Rizzardo et coll. in ACS Symposium Series 1998, 685, 332 have tested various nitroxides, such as 2,2,6,6-tetramethyl-1-piperidinyloxy (known by the abbreviation TEMPO) of the following formula:

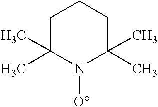

as a control agent within the context of the methyl methacrylate polymerization.

The authors were able to observe that, whichever nitroxide was used within the context of their studies, the polymerization was blocked at a degree of conversion of 30-40% and had no living character. Without being tied to a theory, they attributed this to a secondary dismutation reaction being produced between the nitroxide and the growing macroradical to give a hydroxylamine and polymethyl methacrylate terminated by a carbon-carbon double bond.

There is, therefore a real need for a method for the controlled radical polymerization of methacrylic and/or methacrylate monomers making it possible to:
reach Degree of conversions higher than those encountered in the prior art;
confer a living character on the growing macroradicals, thus enabling a resumption of the polymerization and optionally the synthesis of block copolymers;
obtain polymers or copolymers of high average molecular weight (which may reach more than 80000 g/mol) and of low polydispersity index.

The Applicant has discovered, surprisingly, that by using particular control agents, it is possible to use a polymerization method having the abovementioned characteristics.

### SUMMARY OF THE INVENTION

Thus, the invention relates, according to a first subject matter, to a method for the polymerization of one or more methacrylate and/or methacrylic monomers, for the synthesis of methacrylate or methacrylic polymers or of exclusively methacrylic and/or methacrylate copolymers, comprising a step of bringing said monomer(s) into contact with one at least of the compounds of formula (I) and (II) below:

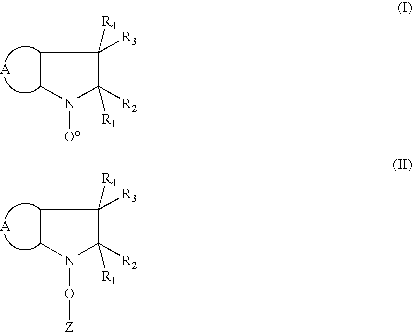

in which:

A represents a hydrocarbon-based group forming an aromatic ring with the two carbon atoms to which it is linked, this ring may bear substituents or may bear one or more, optionally substituted, aromatic or aliphatic fused rings;

R₁, R₂, R₃ and R₄, being identical or different, represent, each independently, an alkyl group, an alkenyl group, an aryl group, an —OH group, an —OR₅ group with R₅ representing an alkyl, alkenyl, aryl or aralkyl group, a —COOH group, a —COOR₆ group with R₆ representing an alkyl group, an alkenyl group, an aryl group or an aralkyl group; or a —CN group; R₃ and R₄ may also be a hydrogen atom or R₃ and R₄ may be joined by ═X, X representing O or NR₇, R₇ representing an alkyl, alkenyl, aryl or aralkyl group;

the alkyl, alkenyl, aryl or aralkyl groups included in the definition of R₁ to R₇ may comprise one or more substituents;

Z represents a group of formula —CR₈R₉R₁₀, in which R₈ and R₉ represent alkyl groups, R₁₀ represents an alkenyl, aryl, aralkyl, CN or COOR₁₁ group, with R₁₁ representing H, Li, Na, K, NH₄⁺, an alkyl, alkenyl, aryl or aralkyl group; and

the alkyl, alkenyl, aryl, aralkyl groups included in the definition of R₈ to R₁₁ may comprise one or more substituents.

Before going into more detail in the description, the following definitions are provided.

The term “methacrylic or methacrylate polymer” is understood to mean a polymer made by the linking of monomer units derived from a methacrylic monomer or from a methacrylate monomer.

The term “exclusively methacrylic and/or methacrylate copolymer” is understood to mean a copolymer made by linking monomer units derived solely from several methacrylic and/or methacrylate monomers.

The term “alkyl group” is understood generally to mean a linear or branched alkyl group comprising from 1 to 20 carbon atoms, or cyclic alkyl group comprising from 3 to 20 carbon atoms. Mention may be made, among these groups, of the methyl, ethyl, n-propyl, i-propyl, n-butyl, n-dodecanyl, i-butyl, t-butyl, cyclopropyl or cyclohexyl group.

The term “aryl group” is understood generally to mean an aromatic group comprising from 6 to 20 carbon atoms. Mention may be made, among these groups, of the phenyl, naphthyl, tolyl or biphenyl group.

The term “aralkyl group” is understood generally to mean an aryl group of the same definition as that given previously, said group being substituted by at least one alkyl group as defined previously, such as a 2-phenylethyl, t-butylbenzyl or benzyl group.

The term “alkenyl group” is understood to generally mean a linear or branched alkenyl group comprising from 2 to 20 carbon atoms, or cyclic alkenyl group comprising from 3 to 20 carbon atoms.

Examples of alkenyl groups are the vinyl, allyl and cyclohexenyl groups.

It is stated that, when the various alkyl, alkenyl, aryl or aralkyl groups comprise one or more substituents, these substituents may be chosen, for example, from halogen atoms, alcohol, ether, amine, carboxylic acid, ester, nitrile, amide, nitro, thiol, thioester, silyl, phosphine or phosphoryl groups.

According to the invention, A represents a hydrocarbon-based group forming an aromatic ring with the two carbon atoms to which it is linked, this ring may bear substituents or may bear one or more, optionally substituted, aromatic or aliphatic fused rings. In other words, the group A may represent an aryl group such as defined above, namely an aromatic group which may comprise from 6 to 20 carbon atoms. It may comprise a single ring (such as a phenyl group), optionally substituted by substituents chosen, for example, from halogen atoms, alcohol, ether, amine, carboxylic acid, ester, nitrile, amide, nitro, thiol, thioester, silyl, phosphine or phosphoryl groups. It may also comprise several rings, of which one at least is an aromatic group, each of the rings may themselves be substituted by substituents chosen from halogen atoms, alcohol, ether, amine, carboxylic acid, ester, nitrile, amide, nitro, thiol, thioester, silyl, phosphine or phosphoryl groups.

Among the compounds of formula (I), mention may advantageously be made of the compounds for which A represents a phenyl group, said compounds corresponding to the formula (Ia) below:

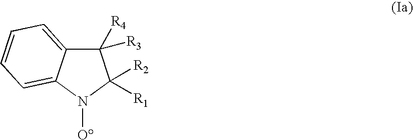

in which R₁, R₂, R₃ and R₄ are as defined above.

Among the compounds of formula (Ia), mention may be made of the compound in which R₁ and R₂ represent an aryl group, R₃ and R₄ together form a group of formula ═N—Ar, the compound thus corresponding to the formula (Iₐ₁) below:

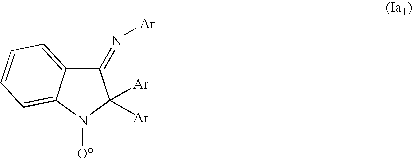

Ar representing an aryl group as defined above, for example, a phenyl group.

Among the compounds of formula (II), mention may advantageously be made of the compounds for which A represents a phenyl group and Z represents a —CR₈R₉R₁₀ group, said compounds thus corresponding to the formula (IIa) below:

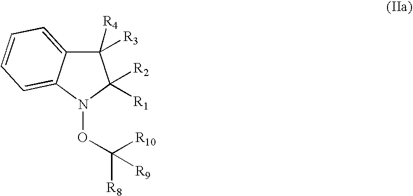

in which R₁, R₂, R₃, R₄, R₈, R₉ and R₁₀ are as defined above.

As an example of compounds of formula (Ia), mention may be made of the particular compound of formula (IIₐ₁) below:

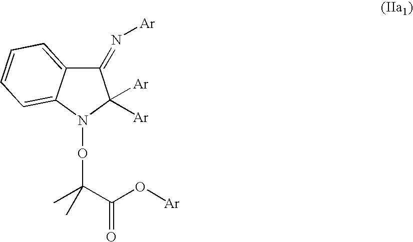

in which the Ar groups, being identical or different, represent an aryl group as defined above.

More precisely, Ar may be a phenyl group, optionally substituted by an —NO₂ group.

Specific compounds corresponding to the definition given above are the following:

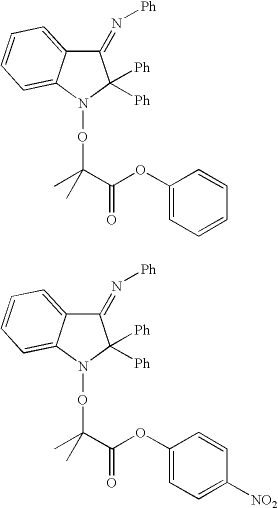

in which Ph represents a phenyl group.

The compound(s) of formula (I) and/or the compound(s) of formula (II) may be present in a content level ranging from 0.005% to 10% by weight relative to the total weight of the monomer(s).

According to one embodiment of the invention, the method, especially when it uses compounds of formula (I), may also be implemented in the presence of a free-radical initiator chosen from hydroperoxides, dialkyl peroxides, diacyl peroxides, peroxyesters, peroxydicarbonates, peroxyacetals or azo compounds.

As examples of hydroperoxides, mention may be made of tert-butyl hydroperoxide, tert-amyl hydroperoxide, cumyl hydroperoxide, 2,5-dimethyl-2,5-di(hydroperoxy)hexane, diisopropylbenzene monohydroperoxide and paramenthane hydroperoxide.

As examples of dialkyl peroxides, mention may be made of 2,5-dimethyl-2,5-di(tert-butylperoxy)-3-hexyne, di-tert-butyl peroxide, di-tert-amyl peroxide, 1,3-di(tert-butylperoxyisopropyl)benzene, 2,5-dimethyl-2,5-di(tert-butylperoxy)hexyne, 1,1,4,4,7,7-hexamethylcyclo-4,7-diperoxynonane and 3,3,6,6,9,9-hexamethylcyclo-1,2,4,5-tetraoxanonane.

As examples of diacyl peroxides, mention may be made of benzoyl peroxide, lauroyl peroxide, decanoyl peroxide, 3,5,5-trimethylhexanoyl peroxide and acetyl-cyclohexyl sulphonyl peroxide.

As examples of peroxyesters, mention may be made of tert-butyl peroxybenzoate, tert-butyl peroxyacetate, tert-butyl peroxy-3,5,5-trimethyl-hexanoate, tert-amyl peroxy-3,5,5-trimethylhexanoate, 2,5-dimethyl-2,5-di(benzoylperoxy)hexane, OO-tert-butyl-O-isopropyl monoperoxycarbonate, OO-tert-butyl-O-(2-ethylhexyl) monoperoxycarbonate, OO-tert-amyl-O-(2-ethylhexyl) monoperoxycarbonate, tert-butyl peroxyisobutyrate, tert-butyl peroxy-2-ethylhexanoate, tert-amyl peroxy-2-ethylhexanoate, 2,5-dimethyl-2,5-di(2-ethylhexanoylperoxy)hexane, tert-butyl peroxyneodecanoate, tert-butyl peroxyisononanoate, tert-butyl peroxypivalate, tert-amyl peroxypivalate, α-cumyl peroxyneodecanoate, tert-amyl peroxydecanoate, tert-butyl 3-hydroxy-1,1-dimethylbutylperoxyneodecanoate and tert-butyl peroxymaleate.

As examples of peroxydicarbonates, mention may be made of di(2-ethylhexyl) peroxydicarbonate, dicyclohexyl peroxydicarbonate, di(n-propyl) peroxydicarbonate, di(4-tert-butylcyclohexyl) peroxydicarbonate.

As examples of peroxyacetals, mention may be made of 1,1-di(tert-butylperoxy)cyclohexane, 1,1-di(tert-butylperoxy)-3,3,5-trimethylcyclohexane, ethyl 3,3-di(tert-butylperoxy)butyrate, ethyl 3,3-di(tert-amylperoxy)butyrate, n-butyl 4,4-di(tert-butylperoxy)valerate, 2,2-di(tert-butylperoxy)butane, 1,1-di(tert-amylperoxy)cyclohexane, 2,2-bis[4,4-di(tert-butylperoxy)cyclohexyl]propane.

Preferably, the free-radical initiator is chosen from the group composed of peroxyesters (such as tert-butyl peroxyneodecanoate, tert-amyl peroxyneodecanoate, tert-butyl peroxypivalate, α-cumyl peroxyneodecanoate), peroxydicarbonates (such as di(2-ethylhexyl) peroxydicarbonate, di(n-propyl) peroxydicarbonate, dicyclohexyl peroxydicarbonate, di(4-tert-butylcyclohexyl) peroxydicarbonate.

The molar ratio of the compound(s) of formula (I) and/or (II) to the free-radical initiator may range from 0.05 to 1.2.

In accordance with the invention, the method relates to the preparation of methacrylate or methacrylic polymers or of copolymers comprising exclusively methacrylate and/or methacrylic units.

The monomer(s) capable of being incorporated into the composition of the polymers or copolymers prepared according to the method of the invention may be chosen from:

methacrylic acid and salts thereof;

C₁-C₁₈ alkyl methacrylates, such as methyl methacrylate and lauryl methacrylate;

C₅-C₁₈ cycloalkyl methacrylates, such as cyclohexyl methacrylate;

alkenyl methacrylates, such as allyl methacrylate;

aryl methacrylates, such as phenyl methacrylate;

hydroxyalkyl methacrylates, such as 2-hydroxyethyl methacrylate and 2-hydroxypropyl methacrylate;

alkyl ether methacrylates, such as 2-ethoxyethyl methacrylate;

alkoxy- or aryloxy-polyalkylene glycol methacrylates, such as methoxypolyethylene glycol methacrylates, ethoxypolyethylene glycol methacrylates, methoxypolypropylene glycol methacrylates and methoxypolyethylene glycol-polypropylene glycol methacrylates;

aminoalkyl methacrylates, such as 2-(dimethylamino)ethyl methacrylate;

methacrylates of amine salts, such as [2-(methacryloyloxy)ethyl]trimethyl ammonium chloride or sulphate, [2-(methacryloyloxy)ethyl]dimethylbenzyl ammonium chloride or sulphate;

fluoro methacrylates, such as 2,2,2-trifluoroethyl methacrylate;

silyl methacrylates, such as 3-methacryloylpropyltrimethylsilane;

phosphorus-containing methacrylates, such as ethylene glycol phosphate methacrylates;

hydroxyethylimidazolidone methacrylate, hydroxyethylimidazolidinone methacrylate, 2-(2-oxo-1-imidazolinyl)ethyl methacrylate; and

mixtures thereof.

The method of the invention applies, more particularly, to the preparation of polymethyl methacrylate.

The method of the invention may apply to bulk polymerization, polymerization in an organic solvent, emulsion polymerization or suspension polymerization methods.

The organic solvent, when it is needed for implementation of a polymerization method, may be chosen from toluene, xylene, chloroform, ethyl acetate, methyl ethyl ketone, dioxane, tetrahydrofuran or dimethylformamide.

The method of the invention is generally conducted at a pressure which may range from 0.5 to 20 bar and at a temperature which may range from 50 to 180° C., and preferably from 90 to 110° C.

The method of the invention may also comprise a step of isolating the polymer or copolymer, for example, by precipitation followed by a filtration. The isolated polymer or copolymer may be used directly for a given application, or may be reintroduced subsequently into a polymerization medium with a view to the resumption of the polymerization (this alternative made possible by the living character of the polymer or copolymer obtained).

In summary, the use of compounds of formulae (I) and (II) has many advantages:

they make it possible to obtain a living polymer or copolymer, the polymerization of which may be restarted either so as to increase the molecular weight of said polymer or copolymer, or in order to synthesize a block copolymer by introduction of another methacrylic and/or methacrylate monomer into the same polymerization conditions as those of the living polymer or copolymer synthesized in the first place;

they allow a polymer or copolymer to be obtained with good control of the polydispersity index.

From a structural point of view, the method of the invention makes it possible to obtain polymers or copolymers having at least one reactive end group having the following formula:

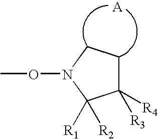

this end group being derived from the combination of compounds of formula (I), introduced in free form or derived from the homolysis of compounds of formula (II), with a radical end group of the synthesized polymer or copolymer, the other end group corresponding to the group -Z, when the control agent used is the compound of formula (II).

This polymer or copolymer having such reactive end groups may be restarted in a polymerization reaction by means of heating (which allows the living character of the polymer or copolymer to be rendered by homolytic cleavage of the reactive end groups) and an optional addition of monomers if there are none left in the medium.

This polymer or copolymer having such reactive end groups may also be made to undergo a chemical transformation of these end groups, by reacting them with suitable reagents.

By way of example, when the compound used corresponds to the following formula:

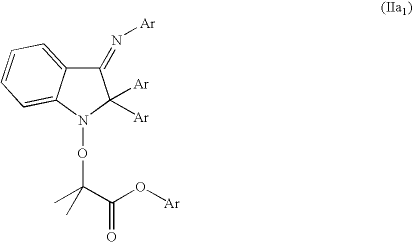

the polymer or copolymer derived from the method of the invention may correspond to the following formula:

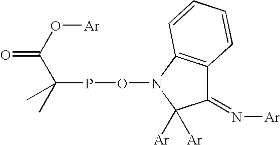

in which P represents the main chain of the polymer or copolymer made by the linking of methacrylic and/or methacrylate monomer units.

After hydrolysis of the —O—Ar bond, the —COOH group thus liberated may be subjected, for example, to esterification reactions or else amidification reactions.

Such reactions may be taken advantage of, for example, for condensing polymers which are not obtained by radical polymerization such as polyesters, polyamides, polyethyleneimines or polyepoxides. These reactions thus allow multiple copolymer structures to be attained.

The polymers or copolymers synthesized by the method of the invention, thanks to the nature of their reactive end groups, are therefore novel.

Thus, the invention relates, according to a second subject matter, to a methacrylic or methacrylate polymer, or an exclusively methacrylic and/or methacrylate copolymer, capable of being obtained by a method as defined above.

In particular, the invention relates to a polymer or copolymer comprising at least one end group of formula:

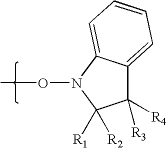

with R₁ and R₂ which may represent a phenyl group, and R₃ and R₄ which may together form an ═N-Ph group, and optionally an end group which may correspond to the formula:

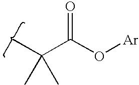

Ar may represent a phenyl group or a 4-nitrophenyl group.

It is stated that the break lines indicate the place where the end groups are joined to the polymer or copolymer.

These polymers or copolymers find their application in all the fields of application for methacrylic and/or methacrylate polymers or copolymers, namely materials that can be used in construction, furniture or electronics, materials that can be used as polyelectrolytes and polymer additives that can be used for their properties of modifying the mechanical properties (impact additives) or modifying the rheology (dispersants, thickeners). In particular, these polymers or copolymers are used for their mechanical properties and/or their transparency properties.

These polymers and copolymers defined above may be incorporated into the constitution of numerous compositions such as:
cosmetic compositions comprising, in addition to said polymers or copolymers, a physiologically acceptable medium;
adhesive compositions, said compositions may comprise, moreover, additives such as tackifying resins and plasticizers, such as oils, in which case it will make a hot-melt pressure-sensitive adhesive composition (known by the abbreviation HMPSA); and
thermoplastic compositions, which may comprise, moreover, one or more thermoplastic polymers, such as polymethyl methacrylate, polystyrene and polyvinyl chloride.

Among the compounds used in the method of the invention, certain compounds are novel.

Thus, the invention relates, according to a third subject matter, to compounds of formula (II) below:

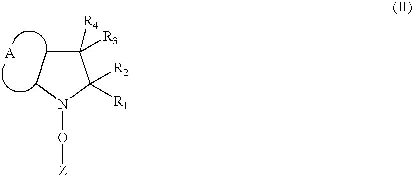

in which A, R₁, R₂, R₃, R₄ and Z are as defined above.

Among the novel compounds of formula (II), mention may be made, advantageously, of the compounds for which A represents a phenyl group and Z represents a —CR₈R₉R₁₀ group, said compounds thus corresponding to the formula (IIa) below:

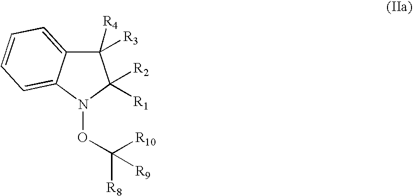

in which R₁, R₂, R₃, R₄, R₉, R₉ and R₁₀ are as defined above.

As an example of compounds of formula (IIa), mention may be made of the particular compound of formula (IIₐ₁) below:

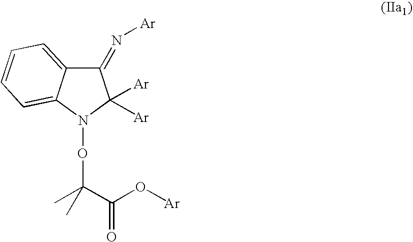

in which the Ar groups, being identical or different, represent an aryl group as defined above.

More precisely, Ar may be a phenyl group, optionally substituted by an —NO₂ group.

Specific compounds corresponding to the definition given above are the following:

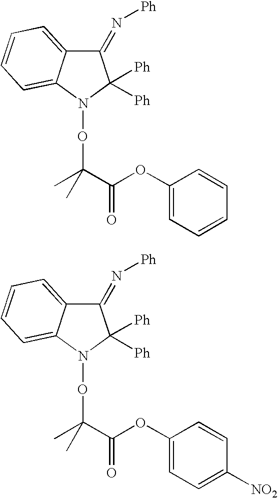

in which Ph represents a phenyl group.

The compounds of formula (II) may be synthesized by a method involving the combination of a carbon-based radical and a nitroxide radical.

Thus, the invention relates, according to a fourth subject matter, to a method of preparing compounds of formula (II), comprising a step consisting in making a nitroxide compound of formula (I) below:

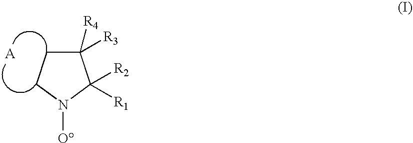

in which A, R₁, R₂, R₃ and R₄ are as defined above;
react with a compound of formula (III) below:

Z-X (III)

in which Z is as defined above and X represents a halogen atom, such as chlorine, fluorine, bromine or iodine;
in a medium comprising at least one organic solvent and one organometallic system comprising a metal salt of formula:

MA

in which:

M represents a transition metal, such as Cu, Ag or Au, and preferably Cu;

A represents a halogen atom; and one organic ligand comprising at least one amine group.

For example, the organic ligand comprising at least one amine group may be chosen from:

tris[2-(dimethylamino)ethyl]amine of the following formula:

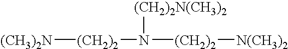

N,N,N′,N′,N″-pentamethyldiethylenetriamine (known by the abbreviation PMDETA) of the following formula:

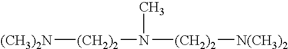

N,N,N′,N′-tetramethylethylenediamine of the following formula:

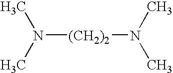

1,1,4,7,10,10-hexamethyltriethylenetetramine (known by the abbreviation HMTETA) of the following formula:

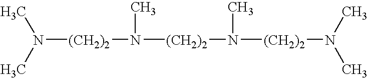

cyclic polyamines, such as:
1,4,7-trimethyl-1,4,7-triazacyclononane;
1,5,9-trimethyl-1,5,9-triazacyclododecane;
1,4,8,11-tetramethyl-1,4,8,1-tetraazacyclotetradecane.

From a practical point of view, in order to obtain compounds of formula (II), generally, a metal salt MA (M and A being as defined above), the organic ligand comprising at least one amine group, the compound of formula (III) and the nitroxide of formula (I), according to a molar ratio of compound (III)/nitroxide (I) ranging from 1 to 1.4, are mixed together with stirring in an organic solvent, while maintaining the reaction medium under stirring at a temperature between 20° C. and 40° C. until the nitroxide (1) has completely disappeared, then recovering the organic phase which is washed with water, then isolating the compound of formula (II) by evaporation of the organic solvent under reduced pressure.

Preferably aromatic hydrocarbons, such as benzene, toluene and xylenes, alkyl chlorides and, in particular, dichloromethane and/or ethers are used as organic solvents.

The metal salt used is preferably CuBr.

CuBr (in which the copper has an oxidation state of 1) and copper may also be introduced into the reaction medium.

The invention will now be described relative to the following examples, given by way of illustration and being nonlimiting.

### DETAILED SUMMARY OF PARTICULAR EMBODIMENTS

### Example 1

This example illustrates the preparation of an alkoxyamine of the following formula:

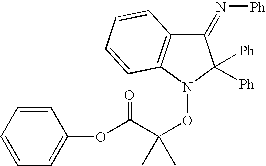

A nitrogen-purged reactor was charged with 10 ml of toluene, 579 mg of N,N,N′,N′,N″-pentamethyldiethylenetriamine (3.6 mmol), 240 mg of CuBr (1.8 mmol) and 106 mg of copper powder (1.8 mmol). 406 mg of 2-bromo-2-methylpropionic acid phenyl ester (1.8 mmol) and 418 mg of DPAIO nitroxide (1.2 mmol) dissolved in 10 ml of toluene were added, said DPAIO nitroxide corresponding to the following formula:

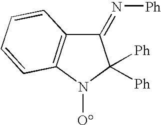

The DPAIO nitroxide was prepared in accordance with that which was explained in Tetraedron, 1975, 31, 1745.

The mixture was left to react for 24 hours at room temperature. The reaction mixture was filtered through celite, then washed with water (4×20 ml) to remove the copper complexes. The organic phase was dried over magnesium sulphate, then evaporated under vacuum. The product was purified by chromatography through a silica column (9:1 pentane/diethyl ether eluent). 460 mg of the compound was obtained in the form of a yellow powder, (yield=75%).

The analytical characteristics of the compound were the following:

¹H NMR (CDCl₃, 300.13 MHz): 1.30 ppm (s, 6H); 6.31 (d, J (H, H)=7.74 Hz, 1H); 6.56 (t, J (H, H)=7.56 Hz, 1H); 6.68 (d, J (H, H)=7.93 Hz, 2H); 6.81 (d, J (H, H)=8.12 Hz, 2H); 6.99 (t, J (H, H)=7.37 Hz, 1H); 7.12 (m, 1H); 7.18 to 7.31 (m, 12H); 7.55 (m, 4H).

Elemental analysis obtained (%): C=80.55; H=5.81; N=5.06 (calculated C=80.27; H=5.61; N=5.20).

### Example 2

This example illustrates the preparation of an alkoxyamine of the following formula:

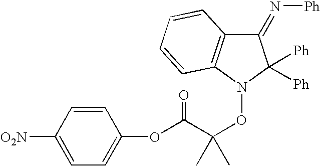

A nitrogen-purged reactor was charged with 40 ml of toluene, 1.34 g of N,N,N′,N′,N″-pentamethyldiethylenetriamine (15.42 mmol), 1.11 g of CuBr (7.71 mmol) and 0.49 g of copper powder (7.71 mmol). 2.22 g of 2-bromo-2-methylpropionic acid 4-nitrophenyl ester (7.71 mmol) and 1.93 mg of DPAIO nitroxide (1.2 mmol) dissolved in 40 ml of toluene were added, said DPAIO nitroxide corresponding to the following formula:

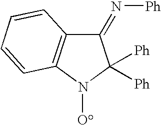

The mixture was left to react for 24 hours at room temperature. The reaction mixture was filtered through celite, then washed with water (4×20 ml) to remove the copper complexes. The organic phase was dried over magnesium sulphate, then evaporated under vacuum. The product was purified by chromatography through a silica column (4:1 pentane/diethyl ether eluent). 1.8 g of the compound was obtained in the form of a yellow powder, (yield=60%).

The analytical characteristics of the compound were the following:

¹H NMR (CDCl₃, 300.13 MHz): 1.46 ppm (s, 6H); 6.41 (d, J (H, H)=7.83 Hz, 1H); 6.67 (t, J (H, H)=6.57 Hz, 1H); 6.75 (d, J (H, H)=7.45 Hz, 2H); 6.98 (d, J (H, H)=9.1 Hz, 2H); 7.08 (t, J (H, H)=7.33 Hz, 1H); 7.29 to 7.82 (m, 10H); 7.61 (m, 4H); 8.18 (d, J (H, H)=9.1 Hz, 2H).

Elemental analysis obtained (%): C=73.64; H=5.13; N=7.12 (calculated C=74.09; H=5.01; N=7.20).

### Example 3

This example illustrates the preparation of a polymethyl methacrylate in a medium comprising:
a DPAIO compound;
a free-radical initiator: di(4-tert-butylcyclohexyl) peroxydicarbonate.

Introduced into a 100 ml Parr reactor were: 40 g of methyl methacrylate (0.4 mol), 0.376 g of DPAIO (1 mmol) of formula:

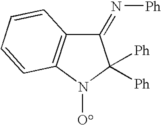

and 0.543 g of di(4-tert-butylcyclohexyl) peroxydicarbonate (Perkadox 16 from Akzo—1.35 mmol). The mixture was degassed by sparging with nitrogen for 30 minutes, then the reactor was sealed and the temperature was raised to 100° C. The Degree of conversions were measured by proton NMR (by comparison of the integrations of the peaks of the vinyl and methyl groups). The number-average and weight-average molecular weights were measured by gel permeation chromatography using a universal calibration and Mark-Houwink parameters (K=0.001298, α=0.688).

The results are given in the following table.

| T | Degree of | Mₙ | Theoretical | Polydispersity |
|---|---|---|---|---|
| (minutes) | conversion (%) | (g/mol) | Mₙ (g/mol) | index, PI |
| 60 | 38 | 15150 | 15200 | 1.80 |
| 120 | 47 | 18350 | 18800 | 1.72 |
| 180 | 52 | 19200 | 20800 | 1.75 |
| 240 | 57 | 20300 | 22800 | 1.78 |

### Example 4

In this example, the same conditions were operated under as in Example 3, except that 0.38 g of di(4-tert-butylcyclohexyl)peroxydicarbonate (0.95 mmol) was used.

The results are given in the following table.

| T | Degree of | Mₙ | Theoretical | Polydispersity |
|---|---|---|---|---|
| (minutes) | conversion (%) | (g/mol) | Mₙ (g/mol) | index, PI |
| 450 | 8 | 8000 | 3200 | 1.15 |
| 1335 | 28 | 35000 | 11200 | 2.25 |
| 1860 | 39 | 44000 | 15600 | 2.4 |
| 2880 | 50 | 48000 | 20000 | 2.5 |
| 3300 | 53 | 62400 | 21200 | 2.4 |
| 4230 | 62 | 68000 | 24800 | 2.4 |

### Example 5

This example illustrates a test of polymerization resumption with methyl methacrylate.

The polymer synthesized in Example 3 was isolated by double precipitation (dissolving in THF, precipitating in ethanol) (Mₙ obtained=30500 g/mol).

7.5 g of the polymer thus obtained (0.25 mmol) was introduced into the Parr reactor in the presence of 60 g of methyl methacrylate (0.6 mol−target weight: 274500 g/mol) and the mixture was raised to a temperature of 100° C.

The results are given in the following table.

| T | Degree of | Mₙ | Theoretical Mₙ |
|---|---|---|---|
| (minutes) | conversion (%) | (g/mol) | (g/mol) |
| 0 | 0 | 30500 | 30500 |
| 90 | 17 | 45800 | 46600 |
| 150 | 20 | 53500 | 54900 |
| 225 | 22 | 61000 | 60400 |
| 285 | 25 | 64820 | 68600 |
| 335 | 26 | 73100 | 71400 |
| 420 | 29 | 82000 | 79600 |

This example illustrates that the polymer obtained in Example 3 partially has a living character and the polymerization may be restarted without adding additional initiator.

It is thus envisageable to synthesize block copolymers thanks to the method of the invention.

### Example 6

Introduced into a 100 ml Parr reactor, were: 40 g of methyl methacrylate (0.4 mol) and 0.54 g of the compound prepared in Example 1 (1 mmol−target weight=40000 g/mol). The mixture was degassed by sparging with nitrogen for 30 minutes, then the temperature was raised to 100° C.

The results are given in the following table.

| T | Degree of | Mₙ | Theoretical | Polydispersity |
|---|---|---|---|---|
| (minutes) | conversion (%) | (g/mol) | Mₙ (g/mol) | index, PI |
| 60 | 9 | 10200 | 3600 | 1.45 |
| 120 | 19 | 11900 | 7600 | 1.37 |
| 180 | 26 | 13350 | 10400 | 1.35 |
| 340 | 41 | 17300 | 16400 | 1.43 |
| 480 | 49 | 20600 | 19600 | 1.41 |
| 600 | 59 | 23500 | 23600 | 1.40 |

This example illustrates that, in the case of the method of the invention, the development of the molecular weights is linear with the Degree of conversion, the weights obtained are close to the theoretical weights and the polydispersity indices are low. This expresses the controlled and living character of the method of the invention.

### Example 7

In this example, the same conditions were operated under as in Example 6, except that 0.27 g of the compound prepared according to Example 1 (0.5 mmol−target weight: 80000 g/mol) were used.

The results are given in the following table.

| T | Degree of | Mₙ | Theoretical | Polydispersity |
|---|---|---|---|---|
| (minutes) | conversion (%) | (g/mol) | Mₙ (g/mol) | index, PI |
| 60 | 12 | 18000 | 9600 | 1.7 |
| 120 | 17 | 21500 | 13600 | 1.6 |
| 180 | 23 | 24000 | 18400 | 1.5 |
| 360 | 33 | 29000 | 26400 | 1.4 |
| 510 | 45 | 36600 | 36000 | 1.7 |
| 1500 | 82 | 58500 | 65600 | 2.1 |

### Example 8

In this example, the same conditions were operated under as in Example 6, except that 0.58 g of the alkoxyamine prepared according to Example 2 (1 mmol−target weight: 40000 g/mol) were used.

The results are given in the following table.

| T | Degree of | Mₙ | Theoretical | Polydispersity |
|---|---|---|---|---|
| (minutes) | conversion (%) | (g/mol) | Mₙ (g/mol) | index, PI |
| 60 | 14 | 6800 | 5600 | 1.32 |
| 120 | 23 | 9950 | 9200 | 1.27 |
| 180 | 29 | 11000 | 11600 | 1.28 |
| 360 | 43 | 19000 | 17200 | 1.42 |
| 580 | 58 | 24500 | 23200 | 1.60 |

### Example 9

In this example, the same conditions were operated under as in Example 6, except that 0.29 g of the alkoxyamine prepared according to Example 2 (0.5 mmol−target weight: 80000 g/mol) were used.

The results are given in the following table.

| T | Degree of | Mₙ | Theoretical | Polydispersity |
|---|---|---|---|---|
| (minutes) | conversion (%) | (g/mol) | Mₙ (g/mol) | index, PI |
| 60 | 11 | 9800 | 8800 | 1.34 |
| 120 | 18 | 15100 | 14400 | 1.30 |
| 180 | 23 | 18700 | 18400 | 1.30 |
| 360 | 33 | 26700 | 26400 | 1.45 |
| 630 | 47 | 33000 | 37600 | 1.80 |

1. Method for the polymerization of one or more methacrylate and/or methacrylic monomers, for the synthesis of methacrylate or methacrylic polymers or methacrylic and/or methacrylate copolymers, comprising a step of contacting said monomer(s) with at least one of the compounds of formula (I) and (II): 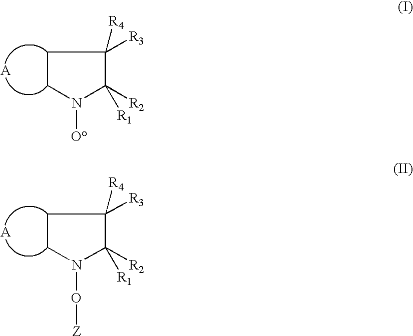
in which:
A represents a hydrocarbon-based group forming an aromatic ring with the two carbon atoms to which it is linked;
R₁, R₂, R₃ and R₄, being identical or different, are chosen from: an alkyl group, an alkenyl group, an aryl group, an —OH group, an —OR₅ group with R₅ representing an alkyl, alkenyl, aryl or aralkyl group, a —COOH group, a —COOR₆ group with R₆ representing an alkyl group, an alkenyl group, an aryl group, an aralkyl group or a —CN group; wherein R₃ and R₄ may further represent a hydrogen atom or be joined by ═X, X representing O or NR₇, R₇ representing an alkyl, alkenyl, aryl or aralkyl group;
; and
Z represents a group of formula —CR₈R₉R₁₀, in which R₈ and R₉ represent alkyl groups, R₁₀ represents an alkenyl, aryl, aralkyl, CN or COOR₁₁ group, with R₁₁ representing H, Li, Na, K, NH₄⁺, an alkyl, alkenyl, aryl or aralkyl group;

2. Method according to claim 1, in which, formula (I), comprises (Ia):

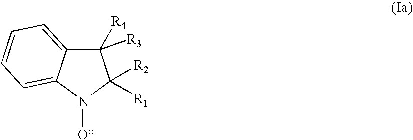

3. Method according to claim 2, in which R₁ and R₂ represent an aryl group, R₃ and R₄ together form a group of formula ═N—Ar, Ar corresponding to an aryl group.

4. Method according to claim 1, in which formula (II) comprises (IIa):

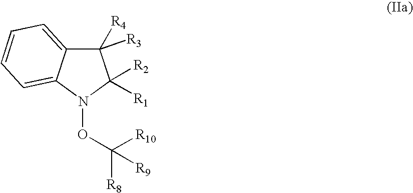

5. Method according to claim 4, in which R₁ and R₂ represent an aryl group, R₃ and R₄ together form a group of formula ═N—Ar, Ar representing an aryl group, R₈ and R₉ represent methyl groups, R₁₀ represents a group of formula —COOAr, Ar representing an aryl group.

6. Method according to claim 4, in which formula (IIa), is chosen from: 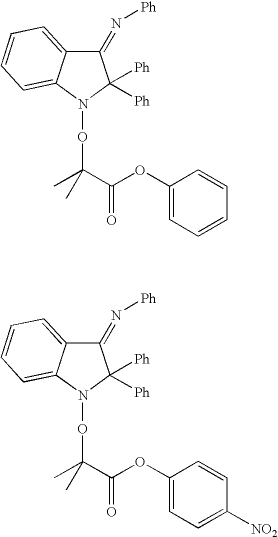
in which Ph represents a phenyl group.

7. Method according to claim 1, in which the contacting step is carried out in the presence of a free-radical initiator chosen from hydroperoxides, dialkyl peroxides, diacyl peroxides, peroxyesters, peroxydicarbonates, peroxyacetals or azo compounds.

8. Method according to claim 7, in which the free-radical initiator is chosen from peroxyesters or peroxydicarbonates.

9. Method according to claim 7, in which the peroxyester is chosen from tert-butyl peroxyneodecanoate, tert-amyl peroxyneodecanoate, tert-butyl peroxypivalate or α-cumyl peroxyneodecanoate.

10. Method according to claim 7, in which the peroxydicarbonate is chosen from di(2-ethylhexyl) peroxydicarbonate, di(n-propyl) peroxydicarbonate, dicyclohexyl peroxydicarbonate or di(4-tert-butylcyclohexyl) peroxydicarbonate.

11. Method according to claim 1, in which the methacrylic and/or methacrylate monomer(s) are chosen from methacrylic acid and salts thereof, C₁-C₁₈ alkyl methacrylates, C₅-C₁₈ cycloalkyl methacrylates, alkenyl methacrylates, aryl methacrylates, hydroxyalkyl methacrylates, alkyl ether methacrylates, alkoxy- or aryloxy-polyalkylene glycol methacrylates, aminoalkyl methacrylates, methacrylates of amine salts, fluoro methacrylates, silyl methacrylates, phosphorus-containing methacrylates, hydroxyethylimidazolidone methacrylate, hydroxyethylimidazolidinone methacrylate, 2-(2-oxo-1-imidazolinyl)ethyl methacrylate; or mixtures thereof.

12. Method according to claim 1, in which the prepared polymer is polymethyl methacrylate.

13. Methacrylate or methacrylic polymer or methacrylic and/or methacrylate copolymer obtained by a method as defined in claim 1.

14. Cosmetic composition comprising, a physiologically acceptable medium and at least one polymer or copolymer as defined in claim 13.

15. Adhesive composition comprising at least one polymer or copolymer as defined in claim 13.

16. Thermoplastic composition comprising at least one polymer or copolymer as defined in claim 13.

17. Compound of formula (II): 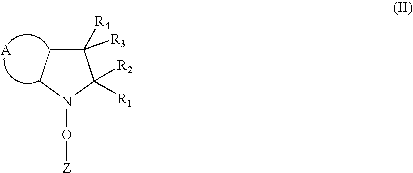
in which:
A represents a hydrocarbon-based group forming an aromatic ring with the two carbon atoms to which it is linked;
R₁, R₂, R₃ and R₄, being identical or different, are selected from the group consisting of, an alkyl group, an alkenyl group, an aryl group, an —OH group, an —OR₅ group with R₅ representing an alkyl, alkenyl, aryl or aralkyl group, a —COOH group, a —COOR₆ group with R₆ representing an alkyl group, an alkenyl group, an aryl group, an aralkyl group or a —CN group; wherein R₃ and R₄ may further represent a hydrogen atom or be joined by ═X, X representing O or NR₇, R₇ representing an alkyl, alkenyl, aryl or aralkyl group;
;
Z represents a group of formula —CR₈R₉R₁₀, in which R₈ and R₉ represent alkyl groups, R₁₀ represents an alkenyl, aryl, aralkyl, CN or COOR₁₁ group, with R₁₁ representing H, Li, Na, K, NH₄⁺, an alkyl, alkenyl, aryl or aralkyl group.
.

18. Compound according to claim 17, corresponding to: 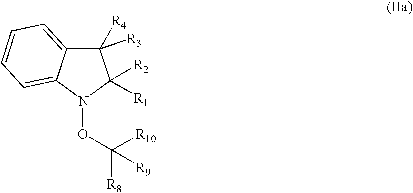
.

19. Compound according to claim 18, in which R₁ and R₂ represent an aryl group, R₃ and R₄ together form a group of formula ═N—Ar, Ar representing an aryl group, R₈ and R₉ represent methyl groups, R₁₀ represents a group of formula —COOAr, Ar representing an aryl group.

20. Compound according to claim 17 chosen from: 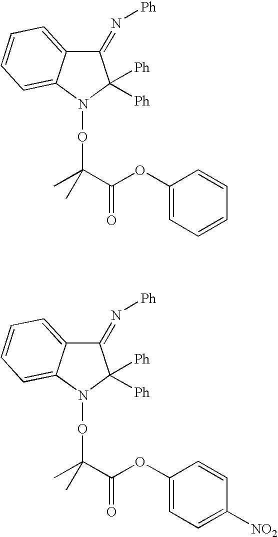
in which Ph represents a phenyl group.

21. Method of preparing a compound of formula (II): 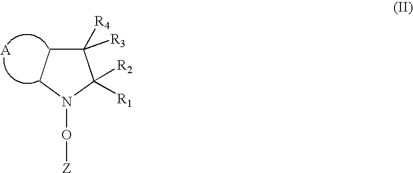
in which:
A represents a hydrocarbon-based group forming an aromatic ring with the two carbon atoms to which it is linked;
R₁, R₂, R₃ and R₄, being identical or different, chosen from, an alkyl group, an alkenyl group, an aryl group, an —OH group, an —OR₅ group with R₅ representing an alkyl, alkenyl, aryl or aralkyl group, a —COOH group, a —COOR₆ group with R₆ representing an alkyl group, an alkenyl group, an aryl group, an aralkyl group or a —CN group; wherein R₃ and R₄ may further represent a hydrogen atom be joined by ═X, X representing O or NR₇, R₇ representing an alkyl, alkenyl, aryl or aralkyl group;
Z represents a group of formula —CR₈R₉R₁₀, in which R₈ and R₉ represent alkyl groups, R₁₀ represents an alkenyl, aryl, aralkyl, CN or COOR₁₁ group, with R₁₁ representing H, Li, Na, K, NH₄⁺, an alkyl, alkenyl, aryl or aralkyl group;
comprising making a nitroxide compound of formula (I) below: 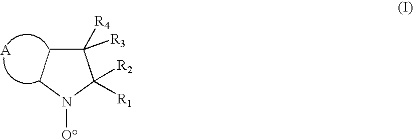
; and
reacting (I) with a compound of formula (III):
Z-X (III)
in which X represents a halogen atom;
in a medium comprising at least one organic solvent and one organometallic system comprising a metal salt of formula:
MA
wherein M represents a transition metal; and
A represents a halogen atom; and
one organic ligand comprising at least one amine group.

22. Method according to claim 21, in which M is Cu.

23. Method according to claim 21, in which the organic ligand comprising at least one amine group is chosen from:
tris[2-(dimethylamino)ethyl]amine of the following formula: 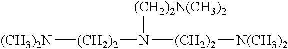
N,N,N′,N′,N″-pentamethyldiethylenetriamine of the following formula: 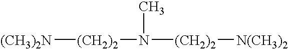
N,N,N′,N′-tetramethylethylenediamine of the following formula: 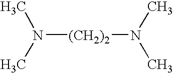
1,1,4,7,10,10-hexamethyltriethylenetetramine (known by the abbreviation HMTETA) of the following formula: 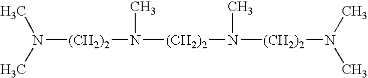
or
cyclic polyamines.

24. Method according to claim 1, in which said aromatic ring may bear substituents or may bear one or more, substituted or un substituted, aromatic or aliphatic fused rings.

25. Method of claim 1, in which the alkyl, alkenyl, aryl or aralkyl groups may comprise one or more substituents.

26. Compound of claim 17, in which said aromatic ring may bear substituents or may bear one or more, substituted or un substituted, aromatic or aliphatic fused rings.

27. Compound of claim 17, in which the alkyl, alkenyl, aryl or aralkyl groups may comprise one or more substituents.

28. Method according to claim 21, in which said aromatic ring may bear substituents or may bear one or more, substituted or un substituted, aromatic or aliphatic fused rings.

29. Method of claim 21, in which the alkyl, alkenyl, aryl or aralkyl groups may comprise one or more substituents.
